(19) United States
(12) Patent Application Publication
(10) Pub. No.: US 2022/0378326 A1
(43) Pub. Date: Dec. 1, 2022

(54) DETECTION DEVICE, DETECTION SYSTEM, AND DETECTION METHOD

(71) Applicant: Panasonic Holdings Corporation, Osaka (JP)

(72) Inventor: Hiroyuki TANI, Hyogo (JP)

(21) Appl. No.: 17/664,685

(22) Filed: May 24, 2022

(30) Foreign Application Priority Data
May 25, 2021 (JP) 2021-087553

(51) Int. Cl. A61B 5/11; A61B 5/00

(52) U.S. Cl. CPC A61B 5/1114; A61B 5/6813; A61B 5/6802; A61B 5/112

(57) ABSTRACT

A detection device, a detection system and a detection method capable of detecting accurate position and orientation of a wearable device on a body are provided. A detection device includes a wireless communicator that receives orientation information indicating an orientation of a wearable device disposed on a body from the wearable device by wireless communication, measures an intensity of a radio wave radiated from the wearable device, and outputs intensity information indicating measured intensity of the radio wave; and a calculator that calculates a position and an orientation of the wearable device on the body based on the orientation information and the intensity information.

### CROSS REFERENCE TO RELATED APPLICATIONS

This application is entitled and claims the benefit of Japanese Patent Application No. 2021-087553, filed on May 25, 2021, the disclosure of which including the specification, drawings and abstract is incorporated herein by reference in its entirety.

### TECHNICAL FIELD

The present disclosure relates to a detection device, a detection system, and a detection method for detecting a position and a direction of a wearable device on a body.

### BACKGROUND ART

Japanese Unexamined Patent Application Publication (Translation of PCT Application) No. 2019-516454 discloses a technique for detecting a position and an orientation of a wearable device on a body, based on a signal indicating a physiological characteristic of the body acquired by the wearable device worn on the body. The signal indicating the physiological characteristic is a signal indicating the heart rate of the body, the skin temperature of the body, the blood pressure of the body, and the like.

The device disclosed in Japanese Unexamined Patent Application Publication (Translation of PCT Application) No. 2019-516454 focuses on association of at least one of the position and the orientation of the wearable device on the body with the physiological characteristic and compares the physiological characteristic with preset data (for example, predetermined characteristics stored in a look-up table). Thus, the device detects the position and orientation of the wearable device on the body suitable for obtaining the physiological properties.

### SUMMARY OF INVENTION

### Technical Problem

The prior art of Japanese Unexamined Patent Application Publication (Translation of PCT Application) No. 2019-516454 is an effective method when the position on the body of the wearable device and the like are related with the physiological characteristics. However, there is a problem that the wearable device that is supposed to be worn at various places on the body may not be able to cope with a desire to detect the accurate position and orientation of the wearable device on the body suitable for obtaining the physiological characteristics.

Non-limiting examples of the present disclosure provide the detection device, the detection system, and the detection method capable of detecting the accurate position and direction of the wearable device on the body.

### Solution to Problem

A detection device according to an embodiment of the present disclosure includes: a wireless communicator that receives orientation information indicating an orientation of a wearable device disposed on a body from the wearable device by wireless communication, measures an intensity of a radio wave radiated from the wearable device, and outputs intensity information indicating measured intensity of the radio wave; and a calculator that calculates a position and an orientation of the wearable device on the body based on the orientation information and the intensity information.

A detection system according to an embodiment of the present disclosure includes: the wearable device; and the detection device according to claim 1, wherein the wearable device comprises: a biometric information sensor that detects biometric information of the body; an orientation detector that detects the orientation on the body; and a wireless communicator that transmits the biometric information and the orientation information detected by the orientation detector to the detection device by wireless communication.

A detection method according to an embodiment of the present disclosure includes: measuring an intensity of a radio wave radiated from a wearable device disposed on a body; receiving orientation information indicating an orientation of the wearable device on the body; estimating a position of the wearable device corresponding to measured intensity of the radio wave; determining whether or not the measured intensity of the radio wave fluctuates for a certain time period; and calculating the orientation of the wearable device at the position on the body of the wearable device based on the orientation information when the intensity of the radio wave does not fluctuate for the certain time period.

### Advantageous Effects of Invention

An embodiment of the present disclosure provides the detection device and the detection method capable of detecting the accurate position and direction of the wearable device on the body.

Further advantages and effects in an embodiment of the present disclosure will be apparent from the specification and drawings. While such advantages and/or effects are respectively provided by the features described in the several embodiments, the specification and drawings, there is no need of providing all of them to obtain one or more of the same features.

### BRIEF DESCRIPTION OF DRAWINGS

FIG. 1 is a diagram illustrating a configuration example of detection system 100 according to an embodiment of the present disclosure;

FIG. 2A is a diagram for explaining a position of wearable device 10 on body 30, the installation position of detection device 20, and the like;

FIG. 2B is a diagram for explaining a position of wearable device 10 on body 30, the installation position of detection device 20, and the like;

FIG. 2C is a diagram for explaining a position of wearable device 10 on body 30, the installation position of detection device 20, and the like;

FIG. 3 is a diagram illustrating a configuration example of wearable device 10;

FIG. 4 is a diagram illustrating a configuration example of detection device 20; and

FIG. 5 is a flowchart for explaining an operation of detection device 20.

### DESCRIPTION OF EMBODIMENTS

Preferred embodiments of the present disclosure will be described in detail with reference to the accompanying drawings. In the present specification and drawings, components having substantially the same functions are denoted by the same reference numerals, and the repetitive description thereof is omitted.

### EMBODIMENTS

### Example of Configuration of Detection System 100

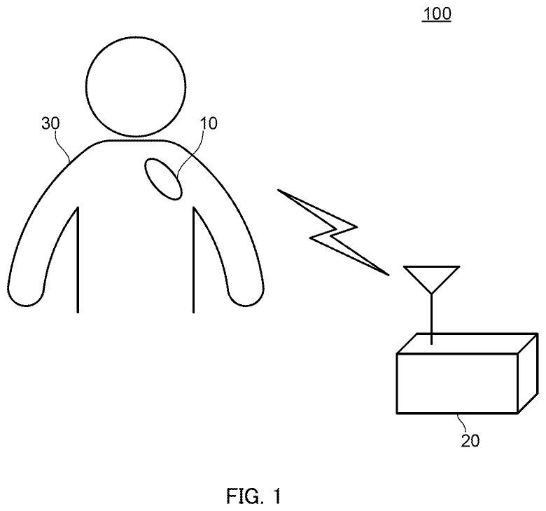

FIG. 1 is a diagram illustrating a configuration example of detection system 100 according to an embodiment of the present disclosure. Detection system 100 is configured to detect the position and direction of wearable device 10 on body 30 even when wearable device 10 is worn anywhere on body 30.

Detection system 100 includes wearable device 10 and detection device 20. Details of the respective configurations of wearable device 10 and detection device 20 will be described later.

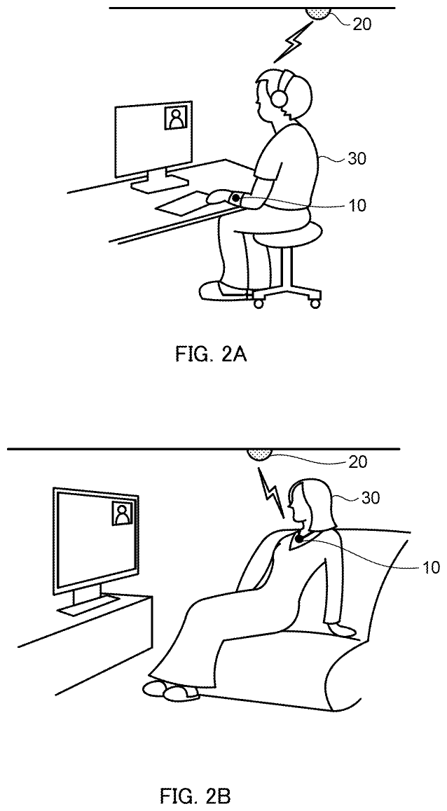

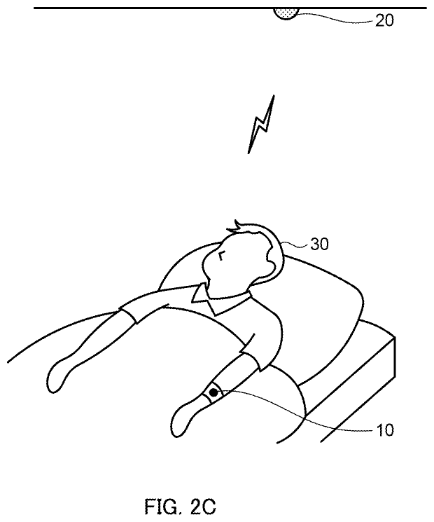

Referring to FIG. 2A, FIG. 2B and FIG. 2C, the position of wearable device 10 on body 30, the installation position of detection device 20 and the like will be described.

FIG. 2A, FIG. 2B, and FIG. 2C are diagrams for explaining the position of wearable device 10 on body 30, the installation position of detection device 20, and the like.

FIG. 2A illustrates a situation where a user wearing wearable device 10 is operating a keyboard installed on a desk while sitting in a chair. In FIG. 2A, wearable device 10 is worn on a wrist of the user, and detection device 20 is installed on a ceiling of a room. In this case, wearable device 10 is assumed to be a watch type capable of wearing on the wrist.

FIG. 2B illustrates a situation where the user wearing wearable device 10 is watching a TV while sitting on a sofa. In FIG. 2B, wearable device 10 is disposed at the chest of the user, and detection device 20 is installed at the ceiling of the room. In this case, wearable device 10 is assumed to be a pendant type capable of hanging on the neck, or incorporating in clothes.

FIG. 2C illustrates a situation where the user wearing wearable device 10 is sleeping in bed. In FIG. 2C, wearable device 10 is worn on the wrist of the user, and detection device 20 is installed on the ceiling of the room. In this case, wearable device 10 is assumed to be a watch type capable of wearing on the wrist.

In this manner, wearable device 10 may be placed at various sites on body 30. The position of wearable device 10 on body 30 and the orientation of wearable device 10 vary depending on the orientation of the part of body 30 where wearable device 10 is placed.

Various types of additional members may be attached to wearable device 10. The additional members may be wristbands, necklace chains, spectacle frames, and the like. By changing the type of such additional members, wearable device 10 can be worn at various positions on body 30.

The installation position of detection device 20 is not limited to the ceiling of the building, and may be on a table, a surface of a wall, or the like.

Although wearable device 10 is attached to body 30 of the human in FIG. 2A and the like, wearable device 10 may be attached to an animal such as a dog, a cat, and a bird in addition to the human.

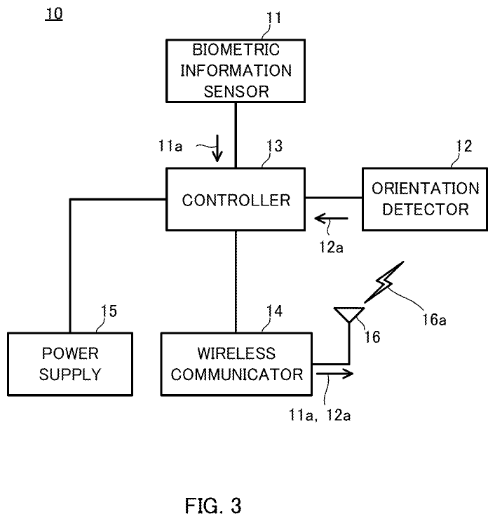

(Wearable Device 10) Next, a configuration example of wearable device 10 will be described with reference to FIG. 3. FIG. 3 is a diagram illustrating a configuration example of wearable device 10.

Wearable device 10 includes biometric information sensor 11, orientation detector 12, controller 13, wireless communicator 14, power supply 15, and antenna 16.

(Biometric Information Sensor 11) Biometric information sensor 11 detects biometric information 11a and sends detected biological information 11a to controller 13. Biometric information 11a includes pulses, brain waves, a muscle potential, presence of sweating, an amount of sweating, a body temperature, a blood pressure, a blood oxygen concentration, and the like.

Biometric information sensor 11 is, for example, an electrocardiographic sensor, an electromyographic sensor, an electroencephalogram sensor, or the like for acquiring such information.

In addition to these sensors, an acceleration sensor, a gyro sensor, a vibration sensor, or the like may be combined with biometric information sensor 11. Thus, it is possible to analyze the state of body 30 of the user wearing wearable device 10 more finely.

(Orientation Detector 12)

Orientation detector 12 detects the orientation of wearable device 10, and transmits orientation information 12a indicating the orientation of wearable device 10 to controller 13.

For example, orientation detector 12 includes a triaxial acceleration sensor (not illustrated). The triaxial acceleration sensor is a sensor for detecting the respective acceleration of the two axes (x-axis, y-axis) direction extending in the horizontal direction, and the acceleration of the one axis (z-axis) direction extending in the vertical direction. The triaxial acceleration sensor detects the inclination of wearable device 10 with respect to the respective extending directions of these three axes, and outputs orientation information 12a indicating the orientation of wearable device 10.

(Controller 13)

Controller 13 includes, for example, electrical circuits such as a memory, a CPU (Central Processing Unit), a communication device, and the like, and controls the operation of wearable device 10 by means of the electric circuits.

Controller 13 stores biometric information 11a transmitted from biometric information sensor 11 and orientation information 12a transmitted from orientation detector 12 in time-series order at predetermined time intervals, and transmits biometric information 11a and orientation information 12a stored in time-series order to wireless communicator 14 sequentially.

(Wireless Communicator 14)

Wireless communicator 14 exchanges information collected by wearable device 10 with detection device 20 in a wireless manner. The wireless communication method may include Bluetooth (registered trademark), RFID (Radio-Frequency Identification), BLE (Bluetooth Low Energy), and the like.

When receiving biometric information 11a and orientation information 12a transmitted from controller 13, wireless communicator 14 transmits biometric information 11a and orientation information 12a to antenna 16.

(Antenna 16)

Antenna 16 receives biometric information 11a and orientation information 12a transmitted from wireless communicator 14, and transmits data including the information in a carrier wave (radio wave 16a) having a predetermined frequency. The type of antenna 16 is, for example, a directional antenna, a non-directional antenna or the like.

(Power Supply 15)

Power supply 15 stores the power necessary for the operation of wearable device 10, and supplies the power to controller 13 and the like.

Power supply 15 includes a battery, an electric circuit for converting the output voltage of the battery into a predetermined voltage level, and a connector for supplying the power to power supply 15. When the battery is a secondary battery, power supply 15 also includes a charging circuit of the secondary battery.

The power supply method to power supply 15 may be, for example, a power supply method by a cable, a non-contact power supply method using a coil, a power supply method by wireless, or the like.

(Detection Device 20)

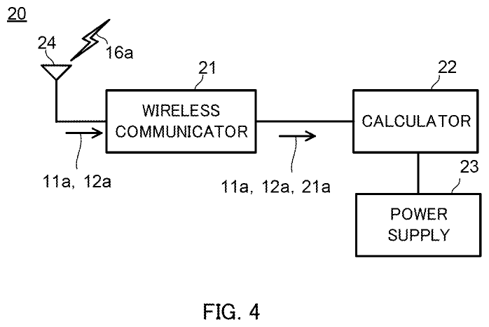

Next, a configuration example of detection device 20 will be described with reference to FIG. 4. FIG. 4 is a diagram illustrating a configuration example of detection device 20. Detection device 20 includes wireless communicator 21, calculator 22, power supply 23 and antenna 24.

(Antenna 24)

Antenna 24 receives radio wave 16a transmitted from wearable device 10 illustrated in FIG. 3, and transmits data included in radio wave 16a to wireless communicator 21. The data includes biometric information 11a and orientation information 12a described above.

(Wireless Communicator 21)

Wireless communicator 21 wirelessly communicates information with wearable device 10. The wireless communication method may include Bluetooth (registered trademark), RFID, BLE, and the like.

Wireless communicator 21 receives the data transmitted from antenna 24, and measures the intensity of radio wave 16a received by antenna 24. By measuring the intensity of radio wave 16a, it is possible to identify the distance from detection device 20 to wearable device 10.

Wireless communicator 21 transmits intensity information 21a indicating the measured intensity of radio wave 16a, together with biometric information 11a and orientation information 12a, to calculator 22.

(Calculator 22)

Detection device 20 specifies the position of wearable device 10 on body 30 based on received intensity information 21a.

Detection device 20 specifies the orientation of wearable device 10 based on orientation information 12a.

(Power Supply 23)

Power supply 23 stores the power necessary for the operation of detection device 20, and supplies the power to calculator 22 and the like.

Power supply 23 includes a battery, an electric circuit for converting the output voltage of the battery into a predetermined voltage level, and a connector for power supply to power supply 23. When the battery is a secondary battery, power supply 23 also includes a charging circuit of the secondary battery.

The power supply method to power supply 23 may be, for example, a power supply method by a cable, a non-contact power supply method using a coil, a power supply method by wireless, or the like.

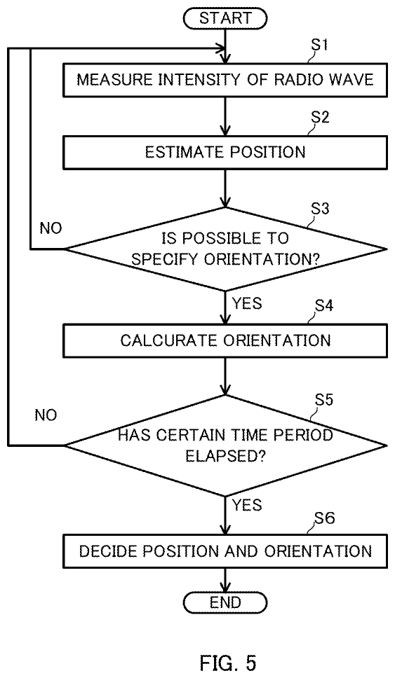

Next, the operation of detection device 20 will be described with reference to FIG. 5. FIG. 5 is a flowchart for explaining the operation of detection device 20.

In step S1, detection device 20 starts wireless communication with wearable device 10, and measures the intensity of radio wave 16a transmitted from wearable device 10.

In step S2, calculator 22 estimate the position of wearable device 10, for example, by referring to the association information for associating the position of wearable device 10 on body 30 with the value of the intensity of radio wave 16a.

In step S3, calculator 22 determines whether or not the intensity of radio wave 16a measured by wireless communicator 21 remains within a predetermined threshold value for a first certain time period.

For example, the threshold of a predetermined width is set in calculator 22. When the intensity of radio wave 16a is not within the threshold value for the first certain time period (for example, several seconds to several tens of seconds), calculator 22 determines that the orientation of wearable device 10 cannot be specified because of the movement of the person being in a non-stationary state, (step S3, NO), and repeats the processes from step S1 to step S3.

When the intensity of radio wave 16a remains within the threshold value for the first certain time period, calculator 22 determines that the orientation of wearable device 10 can be specified because of the movement of the person being in a stationary state (step S3, YES), and executes the process of step S4.

In step S4, calculator 22 calculates the orientation of wearable device 10 at a position on body 30 estimated in step S2 based on orientation information 12a.

Next, in step S5, calculator 22 determines whether or not a second certain time period (for example, several tens of seconds to several minutes) has elapsed from starting the measurement of the intensity of radio wave 16a.

When the second certain time period has not elapsed (step S5, NO), calculator 22 repeats the process from step S1 to step 5 in a predetermined cycle.

Thus, the position of wearable device 10 on body 30 and the orientation of wearable device 10 at the position are corrected.

When the second certain time period has elapsed (step S5, YES), calculator 22 decides the corrected position of wearable device 10 and the orientation of wearable device 10 at the position (step S6), and ends the series of processes.

As described above, the detection apparatus according to the embodiment of the present disclosure includes: a wireless communicator that receives orientation information indicating an orientation of a wearable device disposed on a body from the wearable device by wireless communication, measures an intensity of a radio wave radiated from the wearable device, and outputs intensity information indicating the measured intensity of the radio wave; and a calculator that calculates a position and an orientation of the wearable device on the body based on the orientation information and the intensity information.

With this configuration, the position and orientation of wearable device 10 on body 30 can be detected even if the position and orientation of wearable device 10 on body 30 are not related to physiological characteristics. Accordingly, user-specific biometric information can be analyzed by using wearable device 10 which is assumed to be worn at various locations on the body.

In addition, since the position and orientation of wearable device 10 can be detected at the location distant from wearable device 10, it is also possible to analyze in real time the biological information on body 30 of the user wearing wearable device 10. Thus, it is also possible to recognize the state of the body of the user during the action accurately, and to provide various information according to the state.

While various embodiments have been described above with reference to the drawings, the present disclosure is be limited to such examples. It is apparent that those skilled in the art can make various modifications and variations within the scope of the present disclosure. It is understood that the various modifications and variations are belonging to the scope of the present disclosure. The components in the above embodiments may be combined arbitrarily within the range not deviating from the points of the present disclosure.

As described above, specific examples of the present disclosure have been described in detail. These are just examples and are not intended to limit the scope of the claims. The arts described in the claims may include various modifications and variations of the specific examples exemplified in the present disclosure.

### INDUSTRIAL APPLICABILITY

One embodiment of the present disclosure is suitable for a detection apparatus, a detection system, and a detection method capable of detecting accurate position and orientation of a wearable apparatus on a body.

1. A detection device comprising:
a wireless communicator that receives orientation information indicating an orientation of a wearable device disposed on a body from the wearable device by wireless communication, measures an intensity of a radio wave radiated from the wearable device, and outputs intensity information indicating measured intensity of the radio wave; and
a calculator that calculates a position and an orientation of the wearable device on the body based on the orientation information and the intensity information.

2. The detection device according to claim 1, wherein the calculator estimates the position of the wearable device on the body based on the intensity information and association information indicating association of intensity value of the radio wave measured by the wireless communicator with the position of the wearable device on the body.

3. The detection device according to claim 2, wherein the calculator calculates the orientation of the wearable device on the body based on the orientation information when the intensity of the radio wave measured by the wireless communicator remains within a predetermined width threshold for a first certain time period.

4. The detection device according to claim 2, wherein the calculator determines whether a second certain time period has elapsed from starting measurement of the intensity of the radio wave, and corrects estimated position and calculated orientation when the second certain time period has not elapsed.

5. The detection device according to claim 4, wherein the calculator determines the estimated position and the calculated orientation when the second certain time period has elapsed.

6. A detection system comprising:
the wearable device; and
the detection device according to claim 1,
wherein the wearable device comprises:
a biometric information sensor that detects biometric information of the body;
an orientation detector that detects the orientation on the body; and
a wireless communicator that transmits the biometric information and the orientation information detected by the orientation detector to the detection device by wireless communication.

7. The detection system according to claim 6, wherein the orientation detector comprises a triaxial acceleration sensor.

8. The detection system according to claim 6, wherein the wearable device is configured to be disposed at a plurality of positions on the body.

9. A detection method comprising:
measuring an intensity of a radio wave radiated from a wearable device disposed on a body;
receiving orientation information indicating an orientation of the wearable device on the body;
estimating a position of the wearable device corresponding to measured intensity of the radio wave;
determining whether or not the measured intensity of the radio wave fluctuates for a certain time period; and
calculating the orientation of the wearable device at the position on the body of the wearable device based on the orientation information when the intensity of the radio wave does not fluctuate for the certain time period.
